(19) United States
(12) Patent Application Publication
(10) Pub. No.: US 2011/0003391 A1
(43) Pub. Date: Jan. 6, 2011

(54) SENSOR FILMS, METHODS FOR MAKING AND METHODS FOR MONITORING WATER-SOLUBLE POLYMER CONCENTRATIONS

(76) Inventors: Scott Martell Boyette, New Hope, PA (US); Edward Joseph Schwarz, Haddonfield, NJ (US); Alan Michael Agree, Morrisville, PA (US); Vidyasankar SUNDARESAN, Lansdale, PA (US)

Correspondence Address: General Electric Company;GE Global Patent Operation, 2 Corporate Drive, Suite 648, Shelton, CT, 06484, US

(21) Appl. No.: 12/819,373

(22) Filed: Jun. 21, 2010

Related U.S. Application Data
(60) Provisional application No. 61/222,652, filed on Jul. 2, 2009.

(51) Int. Cl. G01N 33/44; G01N 21/00

(52) U.S. Cl. 436/85; 422/423

(57) ABSTRACT

A multiple-component sensor film includes a carrier polymer matrix and film reactants dispersed within the carrier polymer matrix. The carrier polymer matrix includes a carrier polymer, which includes hydroxypropyl cellulose polymer and the film reactants include an indicator and a reactant from the group of a buffer, a stabilizer, a masking agent, a solubilizer and an internal reference dye. Methods for making the multiple-component sensor film and methods for monitoring the concentration of water soluble polymers in aqueous media are also provided.

### RELATED APPLICATIONS

This application claims priority from U.S. Provisional Application Ser. No. 61/222,652 entitled “Film Sensors and Methods for Making” filed on Jul. 2, 2009, the entirety of which is incorporated.

### FIELD OF THE INVENTION

The present invention relates to sensor films for measuring water-soluble polymers in industrial water systems and, more particularly, for multiple-component sensor films for measuring water-soluble polymers in industrial water systems.

### BACKGROUND OF THE INVENTION

Additives, such as water-soluble polymers, are often added to industrial water systems for inhibiting scale formation on processing equipment. The concentration of the water-soluble polymers can be monitored, such as with an optical sensor, to ensure an optimal concentration range. One type of optical sensor that can be used is a sensor film containing an indicator, which reacts with water-soluble polymers to detect and measure their concentration.

Water-soluble polymers can be very large molecules and cannot readily diffuse into a film for contact with the indicator. Dissolvable sensor films contain a water-soluble matrix and an indicator and can be used for detecting polymers or large molecules. Upon contact with the water to be tested, the water-soluble matrix dissolves and releases the indicator into the water to interact with the water-soluble polymer.

Additional components, such as masking agents, buffer solutions and stabilizers are often needed to optimize detection of the water-soluble polymer. It is difficult to prepare stable multiple-component dissolvable films as the components can interact with one another to form insoluble precipitates and settle or phase separate out of the film mixture.

It would be desirable to obtain a multicomponent dissolvable sensor film for measuring water-soluble polymers in aqueous systems.

### SUMMARY OF THE INVENTION

In one embodiment, a sensor film includes a carrier polymer matrix and film reactants dispersed within the carrier polymer matrix, the carrier polymer matrix includes a carrier polymer including hydroxypropyl cellulose polymer and the film reactants include an indicator and at least one reactant selected from the group consisting of a pH buffer, a stabilizer, a masking agent, an internal reference dye and a solubilizer.

In another embodiment, a method for making a sensor film includes blending an aqueous hydroxypropyl cellulose polymer solution with an indicator to make a first blend, blending an aqueous hydroxypropyl cellulose polymer solution with at least one film reactant selected from the group consisting of a pH buffer, a stabilizer, a masking agent, an internal reference dye and a solubilizer to make a second blend, mixing the first and second blends to make a film solution and forming a film from the film solution.

In another embodiment, a method for monitoring the concentration of water-soluble polymers in aqueous media including contacting the aqueous media with a sensor film, measuring a response from an indicator and equating the response with a concentration amount, wherein the sensor film includes a carrier polymer matrix and film reactants dispersed within the carrier polymer matrix, the carrier polymer matrix including a carrier polymer including hydroxypropyl cellulose polymer and the film reactants including the indicator and at least one film reactant selected from the group consisting of a pH buffer, a stabilizer, a masking agent, an internal reference dye and a solubilizer, whereby the carrier polymer matrix dissolves in the aqueous media and releases the film reactants into the aqueous media.

The various embodiments provide stable multicomponent dissolvable films for detecting and measuring the concentration of water-soluble polymers in industrial water systems.

### BRIEF DESCRIPTION OF DRAWINGS

FIG. 1 is a graph showing the color response for water-soluble polymers relative to amounts of the water-soluble polymers. The graph is color response, which is an absorbance measurement of (Red Absorbance*Yellow Absorbance)/(Green Absorbance squared) vs. the amount of HPS1 or PESA in parts per million by weight (ppm).

### DETAILED DESCRIPTION OF THE INVENTION

The singular forms “a,” “an” and “the” include plural referents unless the context clearly dictates otherwise. The endpoints of all ranges reciting the same characteristic are independently combinable and inclusive of the recited endpoint. All references are incorporated herein by reference.

The modifier “about” used in connection with a quantity is inclusive of the stated value and has the meaning dictated by the context (e.g., includes the tolerance ranges associated with measurement of the particular quantity).

“Optional” or “optionally” means that the subsequently described event or circumstance may or may not occur, or that the subsequently identified material may or may not be present, and that the description includes instances where the event or circumstance occurs or where the material is present, and instances where the event or circumstance does not occur or the material is not present.

In one embodiment, a sensor film includes a carrier polymer matrix and film reactants dispersed within the carrier polymer matrix, the carrier polymer matrix including a carrier polymer including hydroxypropyl cellulose polymer and the film reactants include an indicator and at least one reactant selected from the group consisting of a pH buffer, a stabilizer, a masking agent, an internal reference dye and a solubilizer.

The sensor film can be used in industrial water systems to detect and measure the concentration of water-soluble polymers that may be added to aqueous media in the industrial water systems. The industrial water systems may be cooling towers, boilers, evaporators, gas scrubbers, kilns or desalination units and the aqueous media may be any type of aqueous solutions or water. Water-soluble polymers, including water-soluble biopolymers, may be added to aqueous media in the industrial water systems as dispersants, corrosion inhibitors or other types of additives to protect the processing equipment. In one embodiment, the water-soluble polymers are anionic water-soluble polymers, which contain an anionic group. In one embodiment, the anionic group may include, but is not limited to, carboxylate, sulfonate, sulfate, phosphonate or phosphate. Anionic water-soluble polymers that may be added to aqueous media in the industrial water systems include, but are not limited to, polyacrylic acid and derivatives of polyacrylic acid, polysulfonated polymers or maleic anhydride polymers. In another embodiment, the anionic water-soluble polymers may include poly(acrylic acid-co-1-allyloxy-2-hydroxypropylsulfonate (HPS-1), poly(acrylic acid-co-1-allyloxy-polyethylene oxide sulfate), poly(acrylic acid-co-1-allyloxy-polyethylene oxide sulfate-co-1-allyloxy-2-hydroxy propyl sulfonate) or polyepoxysuccinic acid (PESA).

Many of the water-soluble polymers are large molecules with long chains, which make diffusion into a film difficult. Sensor films are dissolvable films having a water-soluble carrier polymer matrix, which allows the film reactants to interact with the water-soluble polymers in the aqueous media. The carrier polymer matrix includes a hydroxypropyl cellulose polymer (HCP), which is soluble in water and inert to reactions with the water-soluble polymers being detected.

The carrier polymer matrix includes a carrier polymer, which includes HCP. HCP is used to form the carrier polymer matrix for the film and provides support for the film reactants, which are dispersed within the carrier polymer matrix. In one embodiment, the carrier polymer matrix may be formed by any conventional manner. In one embodiment, the carrier polymer matrix may be formed by preparing a film from the HCP. In one embodiment, a film may be made by any means known in the art. Examples of preparing a film are described subsequently.

The film reactants are dispersed within the carrier polymer matrix and are released into the aqueous media being tested when the carrier polymer dissolves upon contact with the aqueous media enabling the film reactants to interact with the water-soluble polymers in the aqueous media. In one embodiment, the film reactants are dispersed within the carrier polymer matrix by blending the film reactants with the carrier polymer. In another embodiment, the film reactants are dispersed within the carrier polymer matrix by blending the film reactants in solution. In another embodiment, the film reactants are blended with the carrier polymer and formed into a film.

In one embodiment, the film reactants include an indicator, which changes its optical properties upon reaction with a water-soluble polymer. In one embodiment, the indicator changes its light absorption or emitting properties and the change can be detected by visible, ultraviolet, infrared or fluorescence absorption, transmission or emission or where changes from clear or transparent to cloudy or opaque can be detected by light scattering.

In one embodiment, the indicator is a dye, such as organic dyes, organic fluorophores, fluorescent dyes, IR absorbing dyes, UV absorbing dyes, photochromic dyes, thermochromic dyes, sulphonephthalein dyes, and other known dyes that may be used for this purpose. In one embodiment, the dyes may include, but are not limited to, bromothymol blue, bromocresol green, bromopyrogallol red, xylidyl blue I, chlorophosphonazo III, brilliant green, xanthene dyes, such as rhodamine B, rhodamine 6G, eosine, phloxine B and the like, acridine dyes, such as acridine orange, acridine red and the like, azo dyes, such as ethyl red, methyl red and the like, porphyrin dyes, phthalocyanine dyes, cyanine dyes, such as 3,3′-diethylthiacarbocyanine iodide, 3,3′-diethyloxadicarbocyanine iodide and the like, merocyanine dyes, styryl dyes, oxonol dyes, triarylmethane dyes, methylene blue, phenol blue and the like. Examples of IR absorbing dyes include carbon black and Poly(styrenesulfonate)/poly(2,3-dihydrothieno(3,4-b)-1,4-dioxin).

In another embodiment, the dye may be a cationic dye. In one embodiment, the dye may be 1,9-dimethyl methylene blue (DMMB), brilliant crystal blue, Basic Blue 17, New Methylene Blue, Azure B or combinations thereof.

The indicator is present in any amount suitable for detecting a water-soluble polymer. In one embodiment, the indicator is present from about 0.5 moles to about 3.0 moles per mole of the water-soluble polymer. In another embodiment, the indicator is present in an amount of from about 0.1 percent by weight to about 10 percent by weight, based on the weight of the carrier polymer. In another embodiment, the indicator is present in an amount of from about 0.5 percent by weight to about 5 percent by weight, based on the weight of the carrier polymer. In another embodiment, the indicator is present in an amount of from about 0.5 percent by weight to about 3 percent by weight, based on the weight of the carrier polymer.

The film reactants may include additional components to enhance the detection and quantitative measurements of the water-soluble polymers. In one embodiment, the film reactants may include at least one reactant selected from the group consisting of pH buffers, stabilizers, masking agents, internal reference dyes and solubilizers.

Many indicators have specific active pH ranges in which the indicator performance is maximized. The pH buffers may be used to adjust the pH of the aqueous media being analyzed to a specific pH range. The pH buffer may be any type of water-soluble buffer. In one embodiment, the buffer includes, but is not limited to, N-(2-acetamido)-2-aminoethanesulfonic acid, 2-(carbamoylmethylamino)ethanesulfonic acid buffer, boric acid, acetic acid, sodium hydroxide, 2-(N-Morpholino)ethanesulfonic acid hydrate/4-Morpholineethanesulfonic acid, 2,2-Bis(hydroxymethyl)-2,2′,2″-nitrilotriethanol/2-Bis(2-hydroxyethyl)amino-2-(hydroxymethyl)-1,3-propanediol/Bis(2-hydroxyethyl)amino-tris(hydroxymethyl)methane, N-(2-Acetamido)iminodiacetic acid/N-(Carbamoylmethyl)iminodiacetic acid, N-(2-Acetamido)-2-aminoethanesulfonic acid/N-(Carbamoylmethyl)-2-aminoethanesulfonic acid/N-(Carbamoylmethyl)taurine, 1,4-Piperazinediethanesulfonic acid/piperazine-1,4-bis(2-ethanesulfonic acid)/piperazine-N,N′-bis(2-ethanesulfonic acid), β-Hydroxy-4-morpholinepropanesulfonic acid/3-Morpholino-2-hydroxypropanesulfonic acid, 1,3-Bis[tris(hydroxymethyl)methylamino]propane, N,N-Bis(2-hydroxyethyl)-2-aminoethanesulfonic acid/N,N-Bis(2-hydroxyethyl)taurine, 3-(N-Morpholino)propanesulfonic acid/4-Morpholinepropanesulfonic acid, N-[Tris(hydroxymethyl)methyl]-2-aminoethanesulfonic acid/2-[(2-Hydroxy-1,1-bis(hydroxymethyl)ethyl)amino]ethanesulfonic acid, 4-(2-Hydroxyethyl)piperazine-1-ethanesulfonic acid/N-(2-Hydroxyethyl)piperazine-N′-(2-ethanesulfonic acid), 3-(N,N-Bis[2-hydroxyethyl]amino)-2-hydroxypropanesulfonic acid/N,N-Bis(2-hydroxyethyl)-3-amino-2-hydroxypropanesulfonic acid, 4-(N-Morpholino)butanesulfonic acid, 2-Hydroxy-3-[tris(hydroxymethyl)methylamino]-1-propanesulfonic acid/N-[Tris(hydroxymethyl)methyl]-3-amino-2-hydroxypropanesulfonic acid, 2-Amino-2-(hydroxymethyl)-1,3-propanediol/Tris(hydroxymethyl)aminomethane/Trometamol, 4-(2-Hydroxyethyl)piperazine-1-(2-hydroxypropanesulfonic acid) Hydrate, piperazine-1,4-bis(2-hydroxypropanesulfonic acid) dihydrate/piperazine-N,N′-bis(2-hydroxypropanesulfonic acid), Tris-EDTA-Acetate, 4-(2-Hydroxyethyl)-1-piperazinepropanesulfonic acid/4-(2-Hydroxyethyl)piperazine-1-propanesulfonic acid/HEPPS/N-(2-Hydroxyethyl)piperazine-N′-(3-propanesulfonic acid), N-[Tris(hydroxymethyl)methyl]glycine, Diglycine/Glycyl-glycine, N,N-Bis(2-hydroxyethyl)glycine, N-(2-Hydroxyethyl)piperazine-N′-(4-butanesulfonic acid), N-[Tris(hydroxymethyl)methyl]-3-aminopropanesulfonic acid/[(2-Hydroxy-1,1-bis(hydroxymethyl)ethyl)amino]-1-propanesulfonic acid, 2-Amino-2-methyl-1,3-propanediol, N-tris(Hydroxymethyl)methyl-4-aminobutanesulfonic acid, N-(1,1-Dimethyl-2-hydroxyethyl)-3-amino-2-hydroxypropanesulfonic acid, 2-(Cyclohexylamino)ethanesulfonic acid, 3-(Cyclohexylamino)-2-hydroxy-1-propanesulfonic acid, 2-Amino-2-methyl-1-propanol, 3-(Cyclohexylamino)-1-propanesulfonic acid, 4-(Cyclohexylamino)-1-butanesulfonic acid and mixtures thereof.

In one embodiment, a pH buffer is used to adjust the aqueous media to be tested to a pH in the range from about 3 to about 10. In another embodiment, the pH is adjusted in the range of from about 6 and to about 8. In another embodiment, the pH is adjusted to a pH of about 7.

The pH buffer is added in any amount sufficient to adjust the pH of the aqueous media. In one embodiment, the pH buffer is present in an amount of from about 1 percent by weight to about 50 percent by weight, based on the weight of the carrier polymer. In another embodiment, the pH buffer may be present in an amount of from about 10 percent by weight to about 30 percent by weight, based on the weight of the carrier polymer. In another embodiment, the pH buffer may be present in an amount of from about 10 percent by weight to about 20 percent by weight, based on the weight of the carrier polymer.

In one embodiment, the film reactant may include a stabilizer. The stabilizer may be used to maintain the solubility of the film reactants in the aqueous media. Once dispersed within the aqueous media, the film reactants can react with other film reactants or with components in the aqueous media to form insoluble precipitants. A stabilizer inhibits precipitates from forming by associating with any minimally soluble material to form complexes that remain soluble in the aqueous media. In one embodiment, the stabilizer may be Arabic gum. In another embodiment, the amount of stabilizer may be in amount up to about 100 ppm by weight, based on the weight of the carrier polymer. In another embodiment, the stabilizer may be in an amount of from about 10 ppm to about 100 ppm by weight, based on the weight of the carrier polymer. In another embodiment, the stabilizer may be in an amount of from about 30 ppm by weight to about 50 ppm by weight, based on the weight of the carrier polymer.

Other types of additives or other water-soluble polymers may be added to the industrial water systems for protecting the processing equipment or treating the aqueous media. These other additives or other water-soluble polymers can interfere with the measurement of a specific water-soluble polymer by reacting with the indicator and affecting a change in the optical properties. In one embodiment, masking agents are used to block the reactions from other types of additives or water-soluble polymers without inhibiting the reaction between the indicator and a specific water-soluble polymer to be measured.

For example, polyepoxysuccinic acid (PESA) may be added in a mixture with HPS-1 to the aqueous media. Both of these polymers can react with the indicator that is used to measure polymer concentrations and, consequently, the measured change in optical properties of the indicator is a function of the total polymer concentration and not of the HPS-1 concentration alone.

In one embodiment, the masking agent is a salt or a cationic surfactant. In one embodiment, the masking agent includes, but is not limited to, bivalent manganese salt, ferrous salt, calcium salts, zinc salts, quaternary amine surfactant or combinations thereof. In another embodiment, the calcium salt may be calcium chloride.

In one embodiment, the masking agent may be added in any amount suitable for masking the response from another additive. In one embodiment, the masking agent is present in an amount up to about 2000 ppm by weight, based on the weight of the carrier polymer. In another embodiment, the masking agent is from about 20 ppm to about 2000 ppm by weight based on the weight of the carrier polymer. In another embodiment, the masking agent may be present from about 100 ppm to about 1000 ppm by weight, based on the weight of the carrier polymer.

In one embodiment, the film reactant may include an internal reference dye, which helps to offset indicator irregularities. A film may have irregularities in thickness across the film and may contain non-uniform amounts of indicator across the film. An internal reference dye is not reactive with the water-soluble polymers to be measured, but will indicate the presence of extraneous measurements, such as irregularities in the amount of indicator, which can be used to adjust the measurement readings of the water-soluble polymers. The internal reference dye may be any type of internal reference dye known in the art. In one embodiment, the internal reference dye may be tris(2,2-bipyridyl)dichlororuthenium(H) hexahydrate. In another embodiment, the internal reference dye is present in any amount suitable for offsetting irregularities in the film. In another embodiment, the internal reference dye is present from about 0.1 percent by weight to about 2.5 percent by weight, based on the weight of the carrier polymer.

In one embodiment, the film reactant may include a solubilizer to enhance solubilization of the film reactants in the aqueous media. In one embodiment, the solubilizer may be any type of solubilizer known in the art. In another embodiment, the solubilizer may be fine silica. In one embodiment, the solubilizer may be present in any amount suitable for enhancing solubility of the film reactants in the aqueous media. In another embodiment, the solubilizer is present in an amount of from about 0.1 percent by weight to about 5 percent by weight, based on the weight of the carrier polymer.

In another embodiment, a method for making a sensor film including blending an aqueous hydroxypropyl cellulose polymer solution with an indicator to make a first blend, blending an aqueous hydroxypropyl cellulose polymer solution with at least one component selected from the group consisting of a pH buffer, a stabilizer, a masking agent, an internal reference dye and a solubilizer to make a second blend, mixing the first and second blends to make a film solution and forming a film from the film solution. In one embodiment, a first blend is prepared by blending HCP with an indicator. The HCP and indicator are described above. In one embodiment, the HCP and indicator are blended in any suitable manner known in the art. In one embodiment, the HCP and indicator are blended in an aqueous solution. In another embodiment, the aqueous solution is a water solution. In one embodiment, the HCP is present in an aqueous HCP solution from about 1 percent by weight to about 12 percent by weight, based on the total volume of water. In another embodiment, the HCP is present in an aqueous HCP solution from about 5 percent by weight to about 10 percent by weight, based on the total volume of water.

In another embodiment, a second blend is prepared by blending HCP with a film reactant selected from the group consisting of pH buffers, stabilizers, masking agents, internal reference dyes and solubilizers. The HCP and the film reactants are described above. In one embodiment, the HCP and at least one film reactant are blended in any suitable manner known in the art. In one embodiment, the HCP and the film reactant are blended in an aqueous solution. In another embodiment, the aqueous solution is a water solution. In one embodiment, the HCP is present in an aqueous HCP solution from about 1 percent by weight to about 12 percent by weight, based on the total volume of water. In another embodiment, the HCP is present in an aqueous HCP solution from about 5 percent by weight to about 10 percent by weight, based on the total volume of water.

Additional film reactants may also be blended into the second blend. In one embodiment, a pH buffer and a masking agent are blended with the aqueous HCP solution to form a second blend. In another embodiment, a pH buffer and a stabilizer are blended with the aqueous HCP solution to form a second blend. In another embodiment, a masking agent and a stabilizer are blended with the aqueous HCP solution to form a second blend. In another embodiment, a pH buffer, a masking agent and a stabilizer are blended with the aqueous HCP solution to form a second blend.

In one embodiment, the first and second blends are mixed together to form a film solution and may be mixed in any manner known in the art. In one embodiment, the first blend and the second blend are mixed in a 1:1 volume ratio. In another embodiment, the first blend may be present in the film solution from about 40 percent by volume to about 60 percent by volume, based on the total volume of the film solution. In another embodiment, the second blend may be present in the film solution in an amount of from about 60 percent by volume to about 40 percent by volume, based on the total volume of the film solution.

In another embodiment, the film solution is formed into a film. A film may be prepared from the film solution in any suitable manner known in the art. In one embodiment, a film may be made by coating a glass or plastic substrate with the solution and removing the solvent from the coating to produce a dry thin film. In one embodiment, the coating may be applied to the substrate by spin coating or by dip coating or by other means known in the art. In another embodiment, a production slot-die coater may be used.

In one embodiment, the solvent may be removed from the coating by drying the coating or evaporating the solvent to form a film. In one embodiment, the coating is allowed to dry at room temperature in the air or may be heated to a temperature of up to about 90° C. In another embodiment, the coating is heated from about 30° C. to about 90° C. In another embodiment, the coating is heated from about 50° C. to about 70° C. The coating may be dried for any length of time sufficient to evaporate the solvent from the coating. In one embodiment, the coating is dried from about 1 minute to about 5 hours.

In another embodiment, the coating is allowed to dry from about 1 minute to about 1 hour. In another embodiment, the coating is allowed to dry from about 1 minute to about 10 minutes.

In one embodiment, the film thickness is from about 0.1 to about 50 microns. In another embodiment, the film thickness is from about 0.5 to about 20 microns. In another embodiment, the film thickness is from about 1 to about 10 microns. In another embodiment, the film thickness may be from about 3 microns to about 20 microns.

The film can be formed to fit a specific dimension or shape. In one embodiment, the film is formed to fit a testing structure. In another embodiment, the film is incorporated into an optical sensor array system, such as is described in U.S. Published Application No. 2007-0092407, which is incorporated herein by reference.

In another embodiment, a method for monitoring the concentration of water-soluble polymers in aqueous media including contacting the aqueous media with a sensor film, measuring a response from an indicator and equating the response with a concentration amount, wherein the sensor film includes a carrier polymer matrix and film reactants dispersed within the carrier polymer matrix, the carrier polymer matrix including a carrier polymer including hydroxypropyl cellulose polymer, the film reactants including the indicator and at least one film reactant selected from the group consisting of a pH buffer, a stabilizer, a masking agent, an internal reference dye and a solubilizer, whereby the carrier polymer matrix dissolves in the aqueous media and releases the film reactants into the aqueous media.

The sensor film can be used in industrial water systems to detect and measure concentrations of water-soluble polymers in the aqueous media. The concentrations of the water-soluble polymers may be monitored to ensure that desired levels of the water-soluble polymers are maintained. Water-soluble polymers are described above and may be added to aqueous media as additives for industrial water systems. The aqueous media may be any type of water or other aqueous solution.

The sensor film contacts the aqueous media to measure the concentrations of water-soluble polymers that may be in the aqueous media. The sensor film contacts the aqueous media in any suitable manner. In one embodiment, the sensor film is added to the aqueous media. In another embodiment, the sensor film may be placed in an optical sensor system, such as an optical sensor array system, and the aqueous media is added to the optical sensor system in contact with the sensor film. Upon contact with the aqueous media, the sensor film dissolves in the aqueous media and releases the indicator and other film reactants into the aqueous media where the film reactants can interact with water-soluble polymers in the aqueous media.

As the indicator interacts with water soluble polymers in the aqueous media, the indicator changes its optical properties and elicits a response, which can be measured. In one embodiment, the indicator changes its light absorption or emitting properties and the change can be detected and measured by visible, ultraviolet, infrared or fluorescence absorption, transmission or emission or where changes from clear or transparent to cloudy or opaque can be detected by light scattering. In one embodiment, the indicator changes color upon interaction with a water soluble polymer and the color response can be measured with visible absorbance by calculating (Red Absorbance*Yellow Absorbance)/(Green Absorbance squared).

The response measurement may be equated with a concentration amount of the water soluble polymers in the aqueous media. In one embodiment, a calibration curve may be prepared for a selected indicator from known concentration amounts of water soluble polymers to measured indicator responses. In another embodiment, a calibration curve is obtained by measuring the absorbance for a specific indicator's color response to known concentrations of water soluble polymers in aqueous media.

The carrier polymer matrix, carrier polymer, hydroxypropyl cellulose polymer (HCP), film reactants, indicator, pH buffer, stabilizer, masking agent, internal reference dye and solubilizer are described above. The film reactants are dispersed within the carrier polymer matrix. In one embodiment, the film reactants are dispersed within the carrier polymer matrix by blending the film reactants with the carrier polymer. In another embodiment, the film reactants are dispersed within the carrier polymer matrix by blending the film reactants in solution. In another embodiment, the film reactants are blended with the carrier polymer and formed into a film.

Monitoring of the concentrations of water soluble polymers in aqueous media may be obtained continuously or in batch testing.

In order that those skilled in the art will be better able to practice the present disclosure, the following examples are given by way of illustration and not by way of limitation.

### EXAMPLES

### Comparative Example 1

A stock solution of HCP was prepared by adding HCP to deionized water at a 10% w/v ratio and stirred vigorously with a Teflon stirrer for about 24 hours under a nitrogen blanket until the HCP was completely dissolved.

A salt stock solution was prepared by adding 975.5 g of calcium chloride dihydrate to 1500.8 g of deionized water and stirred vigorously for 15 minutes.

39.29 g of DMMB dye, 504.83 g of N-(2-Acetamido)-2-aminoethanesulfonic acid (ACES) buffer and 2217.96 g of the calcium chloride dihyrate stock solution were added to the HCP stock solution and mixed for 24 hours under a nitrogen atmosphere. A mixture of calcium chloride salt and ACES buffer of unknown relative composition precipitated out of solution and could not be redissolved.

### Example 2

A stock solution of HCP was prepared by adding HCP to deionized water at a 10% w/v ratio and stirred vigorously with a Teflon stirrer for about 24 hours until the HCP was completely dissolved. The stock solution was split into two halves in separate reactor vessels that were sparged with nitrogen. Each vessel was equipped with a glass or Teflon stirrer and a nitrogen blanket.

A salt stock solution was prepared by adding 975.5 g of calcium chloride dihydrate to 1500.8 g of deionized water and stirred vigorously for 15 minutes.

39.29 g of DMMB dye was mixed with 18,858.21 g of the HCP stock solution in the first vessel for 6 hours under a nitrogen atmosphere until homogenous to yield a first blend. 504.83 g of N-(2-Acetamido)-2-aminoethanesulfonic acid (ACES) pH buffer was mixed with 16,731.33 g of the HCP stock solution in the second reactor vessel blanketed with nitrogen for 6 hours until homogenous to yield a second blend. 2217.96 g of the calcium chloride dihyrate stock solution was also mixed with the second blend as a masking agent for 2 hours under a nitrogen atmosphere until homogenous. The second blend was charged to the first blend and mixed for 6 hours under a nitrogen atmosphere to yield a film solution.

A dry thin film was prepared from the film solution by coating a Dr. Blade and was dried in an oven at 70° C. for 5 minutes. The film had a thickness of 5.5 micrometers.

### Example 3

Several sensor films were prepared as in Example 2 having thicknesses ranging from 4-20 micrometers, and were used to prepare a calibration curve for quantifying HPS-1. The films were placed in an optical sensor array system and water samples were added to the system with the films for testing. The films dissolved in the water samples and the color response of the dye was measured. Absorbance for each sample was measured with a Biotek Power Wave PS PWA reader. The color response was calculated from the absorbance measurements with the following equation: (Red Absorbance*Yellow Absorbance)/(Green Absorbance squared).

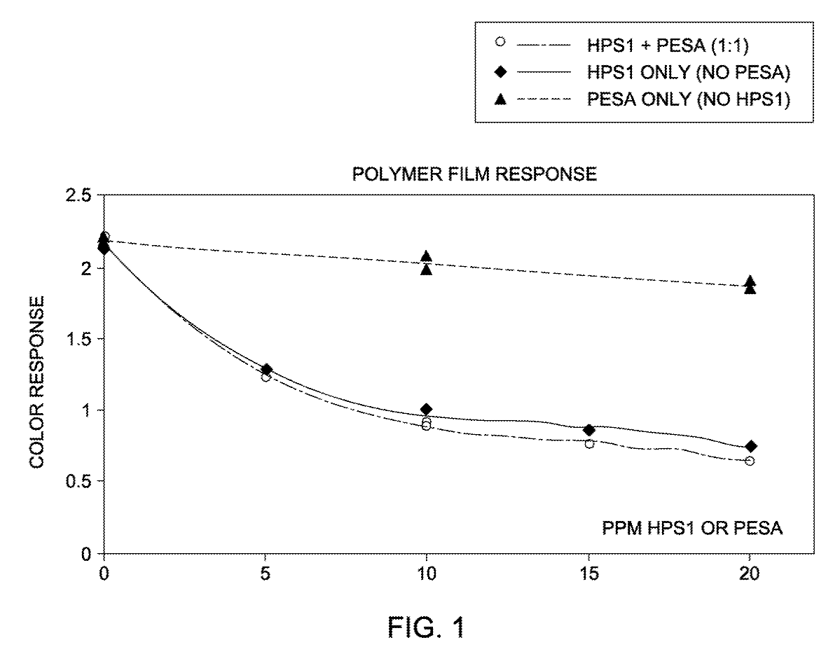

Water samples were prepared by mixing varying amounts of water-soluble polymers in the water. The water samples were (1) blank water samples containing no PESA or HPS-1, (2) water samples containing varying amounts (ppm by weight) of PESA without any HPS-1, (3) water samples containing varying amounts (ppm by weight) of HPS-1 without any PESA, and (4) water samples containing varying amounts (ppm by weight) of a 1:1 by weight blend of HPS-1 and PESA. The polymer film responses for these water samples in groups (2)-(4) were measured against the blank water samples and are shown in FIG. 1.

The film measured the HPS-1 polymer and masked the PESA polymer. There is a slight difference noted in the curves for the water sample containing only HPS-1 and the water sample containing a blend of HPS-1 and PESA, but the difference is well within the limits of statistical error.

While typical embodiments have been set forth for the purpose of illustration, the foregoing descriptions should not be deemed to be a limitation on the scope herein. Accordingly, various modifications, adaptations and alternatives may occur to one skilled in the art without departing from the spirit and scope herein.

Having described the invention that which is claimed is:

1. A sensor film comprising a carrier polymer matrix and film reactants dispersed within the carrier polymer matrix, the carrier polymer matrix comprising a carrier polymer comprising hydroxypropyl cellulose polymer and the film reactants comprising an indicator and at least one reactant selected from the group consisting of a pH buffer, a stabilizer, a masking agent, an internal reference dye and a solubilizer.

2. The sensor film of claim 1 wherein the indicator is a dye.

3. The sensor film of claim 1 wherein the indicator is present in an amount of from about 0.1 percent by weight to about 10 percent by weight, based on the weight of the carrier polymer.

4. The sensor film of claim 1 wherein the pH buffer is selected from the group consisting of N-(2-acetamido)-2-aminoethanesulfonic acid, 2-(carbamoylmethylamino)ethanesulfonic acid buffer, boric acid, acetic acid, sodium hydroxide, 2-(N-Morpholino)ethanesulfonic acid hydrate/4-Morpholineethanesulfonic acid, 2,2-Bis(hydroxymethyl)-2,2′,2″-nitrilotriethanol/2-Bis(2-hydroxyethyl)amino-2-(hydroxymethyl)-1,3-propanediol/Bis(2-hydroxyethyl)amino-tris(hydroxymethyl)methane, N-(2-Acetamido)iminodiacetic acid/N-(Carbamoylmethyl)iminodiacetic acid, N-(2-Acetamido)-2-aminoethanesulfonic acid/N-(Carbamoylmethyl)-2-aminoethanesulfonic acid/N-(Carbamoylmethyl)taurine, 1,4-Piperazinediethanesulfonic acid/Piperazine-1,4-bis(2-ethanesulfonic acid)/Piperazine-N,N′-bis(2-ethanesulfonic acid), P-Hydroxy-4-morpholinepropanesulfonic acid/3-Morpholino-2-hydroxypropanesulfonic acid, 1,3-Bis[tris(hydroxymethyl)methylamino]propane, N,N-Bis(2-hydroxyethyl)-2-aminoethanesulfonic acid/N,N-Bis(2-hydroxyethyl)taurine, 3-(N-Morpholino)propanesulfonic acid/4-Morpholinepropanesulfonic acid, N-[Tris(hydroxymethyl)methyl]-2-aminoethanesulfonic acid/2-[(2-Hydroxy-1,1-bis(hydroxymethyl)ethyl)amino]ethanesulfonic acid, 4-(2-Hydroxyethyl)piperazine-1-ethanesulfonic acid/N-(2-Hydroxyethyl)piperazine-N′-(2-ethanesulfonic acid), 3-(N,N-Bis[2-hydroxyethyl]amino)-2-hydroxypropanesulfonic acid/N,N-Bis(2-hydroxyethyl)-3-amino-2-hydroxypropanesulfonic acid, 4-(N-Morpholino)butanesulfonic acid, 2-Hydroxy-3-[tris(hydroxymethyl)methylamino]-1-propanesulfonic acid/N-[Tris(hydroxymethyl)methyl]-3-amino-2-hydroxypropanesulfonic acid, 2-Amino-2-(hydroxymethyl)-1,3-propanediol/Tris(hydroxymethyl)aminomethane/Trometamol, 4-(2-Hydroxyethyl)piperazine-1-(2-hydroxypropanesulfonic acid)Hydrate, piperazine-1,4-bis(2-hydroxypropanesulfonic acid) dihydrate/Piperazine-N,N′-bis(2-hydroxypropanesulfonic acid), Tris-EDTA-Acetate, 4-(2-Hydroxyethyl)-1-piperazinepropanesulfonic acid/4-(2-Hydroxyethyl)piperazine-1-propanesulfonic acid/HEPPS/N-(2-Hydroxyethyl)piperazine-N′-(3-propanesulfonic acid), N-[Tris(hydroxymethyl)methyl]glycine, Diglycine/Glycyl-glycine, N,N-Bis(2-hydroxyethyl)glycine, N-(2-Hydroxyethyl)piperazine-N′-(4-butanesulfonic acid), N-[Tris(hydroxymethyl)methyl]-3-aminopropanesulfonic acid/[(2-Hydroxy-1,1-bis(hydroxymethyl)ethyl)amino]-1-propanesulfonic acid, 2-Amino-2-methyl-1,3-propanediol, N-tris(Hydroxymethyl)methyl-4-aminobutanesulfonic acid, N-(1,1-Dimethyl-2-hydroxyethyl)-3-amino-2-hydroxypropanesulfonic acid, 2-(Cyclohexylamino)ethanesulfonic acid, 3-(Cyclohexylamino)-2-hydroxy-1-propanesulfonic acid, 2-Amino-2-methyl-1-propanol, 3-(Cyclohexylamino)-1-propanesulfonic acid, 4-(Cyclohexylamino)-1-butanesulfonic acid and mixtures thereof.

5. The sensor film of claim 1 wherein the pH buffer is present in an amount of from about 1 percent by weight to about 50 percent by weight, based on the weight of the carrier polymer.

6. The sensor film of claim 1 wherein the stabilizer is Arabic gum.

7. The sensor film of claim 1 wherein the stabilizer is in amount up to about 100 ppm by weight, based on the weight of the carrier polymer.

8. The sensor film of claim 1 wherein the masking agent is a salt or a cationic surfactant.

9. The sensor film of claim 1 wherein the masking agent is added in an amount up to about 2000 ppm by weight, based on the weight of the carrier polymer.

10. The sensor film of claim 1 wherein the internal reference dye is tris(2,2-bipyridyl)dichlororuthenium(II) hexahydrate.

11. The sensor film of claim 1 wherein the internal reference dye is present from about 0.1 percent by weight to about 2.5 percent by weight, based on the weight of the carrier polymer.

12. The sensor film of claim 1 wherein the solubilizer is fine silica.

13. The sensor film of claim 1 wherein the solubilizer is present in an amount of from about 0.1 percent by weight to about 5 percent by weight, based on the weight of the carrier polymer.

14. The sensor film of claim 1 wherein the thickness of the film is from about 0.1 to about 50 microns.

15. A method for making a sensor film comprising blending an aqueous hydroxypropyl cellulose polymer solution with an indicator to make a first blend, blending an aqueous hydroxypropyl cellulose polymer solution with at least one film reactant selected from the group consisting of a pH buffer, a stabilizer, a masking agent, an internal reference dye and a solubilizer to make a second blend, mixing the first and second blends to make a film solution and forming a film from the film solution.

16. The method of claim 15 wherein the indicator is a dye.

17. The method of claim 15 wherein the indicator is present in an amount of from about 0.1 percent by weight to about 10 percent by weight, based on the weight of the carrier polymer.

18. The method of claim 15 wherein the pH buffer is selected from the group consisting of N-(2-acetamido)-2-aminoethanesulfonic acid, 2-(carbamoylmethylamino)ethanesulfonic acid buffer, boric acid, acetic acid, sodium hydroxide, 2-(N-Morpholino)ethanesulfonic acid hydrate/4-Morpholineethanesulfonic acid, 2,2-Bis(hydroxymethyl)-2,2′,2″-nitrilotriethanol/2-Bis(2-hydroxyethyl)amino-2-(hydroxymethyl)-1,3-propanediol/Bis(2-hydroxyethyl)amino-tris(hydroxymethyl)methane, N-(2-Acetamido)iminodiacetic acid/N-(Carbamoylmethyl)iminodiacetic acid, N-(2-Acetamido)-2-aminoethanesulfonic acid/N-(Carbamoylmethyl)-2-aminoethanesulfonic acid/N-(Carbamoylmethyl)taurine, 1,4-Piperazinediethanesulfonic acid/Piperazine-1,4-bis(2-ethanesulfonic acid)/Piperazine-N,N′-bis(2-ethanesulfonic acid), β-Hydroxy-4-morpholinepropanesulfonic acid/3-Morpholino-2-hydroxypropanesulfonic acid, 1,3-Bis[tris(hydroxymethyl)methylamino]propane, N,N-Bis(2-hydroxyethyl)-2-aminoethanesulfonic acid/N,N-Bis(2-hydroxyethyl)taurine, 3-(N-Morpholino)propanesulfonic acid/4-Morpholinepropanesulfonic acid, N-[Tris(hydroxymethyl)methyl]-2-aminoethanesulfonic acid/2-[(2-Hydroxy-1,1-bis(hydroxymethyl)ethyl)amino]ethanesulfonic acid, 4-(2-Hydroxyethyl)piperazine-1-ethanesulfonic acid/N-(2-Hydroxyethyl)piperazine-N′-(2-ethanesulfonic acid), 3-(N,N-Bis[2-hydroxyethyl]amino)-2-hydroxypropanesulfonic acid/N,N-Bis(2-hydroxyethyl)-3-amino-2-hydroxypropanesulfonic acid, 4-(N-Morpholino)butanesulfonic acid, 2-Hydroxy-3-[tris(hydroxymethyl)methylamino]-1-propanesulfonic acid/N-[Tri s(hydroxymethyl)methyl]-3-amino-2-hydroxypropanesulfonic acid, 2-Amino-2-(hydroxymethyl)-1,3-propanediol/Tris(hydroxymethyl)aminomethane/Trometamol, 4-(2-Hydroxyethyl)piperazine-1-(2-hydroxypropanesulfonic acid)Hydrate, Piperazine-1,4-bis(2-hydroxypropanesulfonic acid) dihydrate/piperazine-N,N′-bis(2-hydroxypropanesulfonic acid), Tris-EDTA-Acetate, 4-(2-Hydroxyethyl)-1-piperazinepropanesulfonic acid/4-(2-Hydroxyethyl)piperazine-1-propanesulfonic acid/HEPPS/N-(2-Hydroxyethyl)piperazine-N′-(3-propanesulfonic acid), N-[Tris(hydroxymethyl)methyl]glycine, Diglycine/Glycyl-glycine, N,N-Bis(2-hydroxyethyl)glycine, N-(2-Hydroxyethyl)piperazine-N′-(4-butanesulfonic acid), N-[Tris(hydroxymethyl)methyl]-3-aminopropanesulfonic acid/[(2-Hydroxy-1,1-bis(hydroxymethyl)ethyl)amino]-1-propanesulfonic acid, 2-Amino-2-methyl-1,3-propanediol, N-tris(Hydroxymethyl)methyl-4-aminobutanesulfonic acid, N-(1,1-Dimethyl-2-hydroxyethyl)-3-amino-2-hydroxypropanesulfonic acid, 2-(Cyclohexylamino)ethanesulfonic acid, 3-(Cyclohexylamino)-2-hydroxy-1-propanesulfonic acid, 2-Amino-2-methyl-1-propanol, 3-(Cyclohexylamino)-1-propanesulfonic acid, 4-(Cyclohexylamino)-1-butanesulfonic acid and mixtures thereof.

19. The method of claim 15 wherein the pH buffer is present in an amount of from about 1 percent by weight to about 50 percent by weight, based on the weight of the carrier polymer.

20. The method of claim 15 wherein the stabilizer is Arabic gum.

21. The method of claim 15 wherein the stabilizer is in amount up to about 100 ppm by weight, based on the weight of the carrier polymer.

22. The method of claim 15 wherein the masking agent is a salt or a cationic surfactant.

23. The method of claim 15 wherein the masking agent is added in an amount up to about 2000 ppm by weight, based on the weight of the carrier polymer.

24. The method of claim 15 wherein the internal reference dye is tris(2,2-(E-bipyridyl)dichlororuthenium(II) hexahydrate.

25. The method of claim 15 wherein the internal reference dye is present from about 0.1 percent by weight to about 2.5 percent by weight, based on the weight of the carrier polymer.

26. The method of claim 15 wherein the solubilizer is fine silica.

27. The method of claim 15 wherein the solubilizer is present in an amount of from about 0.1 percent by weight to about 5 percent by weight, based on the weight of the carrier polymer.

28. The method of claim 15 wherein the first blend is present in the film solution from about 40 percent by volume to about 60 percent by volume, based on the total volume of the film solution and the second blend is present in the film solution in an amount of from about 60 percent by volume to about 40 percent by volume, based on the total volume of the film solution.

29. A method for monitoring the concentration of water-soluble polymers in aqueous media including contacting the aqueous media with a sensor film, measuring a response from an indicator and equating the response with a concentration amount, wherein the sensor film includes a carrier polymer matrix and film reactants dispersed within the carrier polymer matrix, the carrier polymer matrix comprising a carrier polymer comprising hydroxypropyl cellulose polymer, the film reactants including the indicator and at least one film reactant selected from the group consisting of a pH buffer, a stabilizer, a masking agent, an internal reference dye and a solubilizer, whereby the carrier polymer matrix dissolves in the aqueous media and releases the film reactants into the aqueous media.
